(19) United States
(12) Patent Application Publication
(10) Pub. No.: US 2011/0200539 A1
(43) Pub. Date: Aug. 18, 2011

(54) Sprayable composition comprising extract of red vine leaves

(75) Inventors: Katrin Buszello, Birkenhard (DE); Oliver Ludwig Freichel, Gau-Algesheim (DE); Martin Langer, Ulm (DE); Bernd Plohmann, Oberhofen (DE)

(73) Assignee: BOEHRINGER INGELHEIM INTERNATIONAL GMBH, Ingelheim am Rhein (DE)

(21) Appl. No.: 12/674,914

(22) PCT Filed: Aug. 26, 2008
(86) PCT No.: PCT/EP2008/061110

§ 371 Date: May 2, 2011

(30) Foreign Application Priority Data
Aug. 31, 2007 (DE) 102007041556.9

(51) Int. Cl. A61K 9/12; A61P 9/00; A61P 17/00

(52) U.S. Cl. 424/45; 424/43

(57) ABSTRACT

Disclosed are sprayable compositions containing red vine leaf extract, which can be sprayed onto the skin using suitable spray devices.

### BACKGROUND TO THE INVENTION

Many people, especially women, have a tendency to weakness of the veins, which is also known as chronic venous insufficiency (CVI). People with CVI suffer from heavy, swollen legs, especially in warm weather or after sitting or standing for long periods. These symptoms restrict the function of the legs considerably, which in turn has negative effects on mobility and activity and, generally, on the wellbeing of those affected. According to the established categories developed by Widmer, clinically significant CVI is divided into three stages: Stage 1: reversible oedema, corona phlebectatica (dark-blue changes to the veins in the skin on the med. and lat. edges of the feet), perimalleolar varicose veins; Stage 2: persistent oedema, haemosiderosis and purple discoloration of the skin in the lower par of the leg, dermatosclerosis and lipodermatosclerosis, atrophie blanche, stasis eczema, cyanotic skin coloration; Stage 3: leg ulcers.

Extracts of red vine leaves of the species vitis vinifera have been known and used widely for a long time. In traditional or folk medicine, corresponding preparations have long been used to treat a wide range of ailments and pains. Thus, for example, in France, as can be found in the Pharmacopée Française, red vine leaf extract is used as a vein tonic for treating various vein ailments.

Modern preparations based on red vine leaves include, for example, the capsules and tablets for treating chronic venous insufficiency that are marketed by Boehringer Ingelheim under the trade mark Antistax®. These contain a red vine leaf extract with a high content of flavonoids, which according to the findings of medical experts play a crucial role in the sealing of veins and have an anti-inflammatory effect. The above-mentioned capsules and tablets are intended exclusively for oral use.

Also on the market is an Antistax® ointment for topical application to the skin. This ointment also contains red vine leaf extract. However, the ointment has to be applied by hand to the affected areas of the skin, as a result of which, firstly, problems may arise as a result of the application of ointment to areas of the hand and, secondly, the ointment is often applied unevenly.

The problem therefore is to provide a composition containing red vine leaf extract which can be applied evenly and without coming into contact with the surfaces of the user's hands.

### SUMMARY OF THE INVENTION

The invention described here solves the above problem by providing a sprayable composition containing red vine leaf extract, preferably from vitis vinifera L. This can be sprayed onto the skin using suitable spray devices, so as to achieve a relatively uniform application of the composition without it coming into direct contact with the user's hands.

It is known from EP 1 581 239 that aqueous red vine leaf extract has a positive effect on the microcirculation in the capillaries of the lower extremities. This was demonstrated using Laser Doppler Flowmetry (cf. also Tulevski I I, Ubbink D T, Jacobs M J H M. Red and green laser Doppler compared with capillary microscopy to assess skin microcirculation in the feet of healthy subjects. Microvasc Res 1999; 58(2):83-88; and Bollinger A, Jäger K, Jünger M, Seifert H. The vascular laboratory: advances in non-invasive techniques. World J Surg 1988; 12:724-731).

A particularly preferred embodiment of the present invention allows a more uniform application of red vine leaf extract to the skin, with faster, optimum permeation of the flavonoid-like active ingredients, so as to obtain a more uniform and effective activity in the upper skin layers (e.g. in the dermis).

### DETAILED DESCRIPTION OF THE INVENTION

The sprayable composition according to the present invention contains as its main component red vine leaf extract, typically in a concentration of 0.1 wt.-% to 15 wt.-%, preferably from 0.3 wt.-% to 10 wt.-% and particularly preferably from 0.5 wt.-% to 8 wt.-%, in each case based on the dry extract and the total mass of the composition. This is preferably obtained by extraction from the leaves of vitis vinifera L., preferably at a time when the flavonoid concentration in the leaves has reached its optimum level. This is achieved at about the time of the grape harvest. The extraction is preferably carried out as a liquid extraction, i.e. using a liquid extracting agent. The extracting agent used may be any of those which are acceptable in the manufacture of foods or pharmaceuticals. Ethanol and water are preferred, as well as the mixtures thereof. A preferred process for preparing the red vine leaf extract for use within the scope of the present invention will now be described, comprising the following steps:
(a) collecting red vine leaves at a time when the flavonoid content has reached an optimum level;
(b) drying and comminuting the leaves;
(c) cutting the leaves into pieces;
(d) extracting the shredded leaves with water or ethanol or mixtures thereof at elevated temperature for 6 to 10 hours;
(e) optionally concentrating the extract obtained.

When the leaves are cut up in step (c) the pieces obtained preferably range in size from 5 to 10 mm. The extraction in step (d) is preferably carried out at elevated temperature, preferably in the range from 60° C. to 80° C. The concentration of the extract in step (e) is preferably carried out by the removal of moisture in vacuo and may be carried out to dryness.

Within the scope of the present invention it is preferable to use red vine leaf extract which contains flavonoids in the range from 1 to 20 wt.-%, preferably 2 to 10 wt.-%, based on the dry mass of the extract.

In addition to the red vine leaf extract the sprayable composition of the present invention optionally also contains other ingredients. These are preferably propellants, skin care substances and adjuvants.

“Propellants” within the scope of the present invention refers to all cosmetically and pharmaceutically acceptable agents which are suitable for spraying a liquid or emulsion using a suitable spraying device. Preferred propellants are propellant gases that can be liquefied under elevated pressure and are miscible with water under elevated pressure, such as dimethylether, which by virtue of its dissolving properties in aqueous media allows a brief supersaturation of the ingredients, particularly the flavonoids, on the skin during spraying and thus also allows rapid permeation into the upper skin layers. The thermodynamic activity, defined as the concentration of active substance in the vehicle to its saturation solubility, becomes greater than 1, as a result of this principle, so that the amount of active substance absorbed (maximum flux) is greater than in a conventional hydrophilic gel.

In preferred embodiments of the present invention the propellant comprises at least one highly volatile substance with a vapour pressure of typically 1 to 10 bar, preferably 3 to 8 bar and particularly preferably 5.1 bar, in each case at 20° C., in an amount of typically at least 1 wt.-%, preferably at least 5 wt.-%, preferably at least 10 wt.-%, particularly preferably at least 20 wt.-%, in each case based on the total mass of the composition. A preferred example is dimethylether. As a result of their high vapour pressure, highly volatile substances of this kind evaporate rapidly on the user's skin, which is cooled by the cold due to evaporation thus produced. This cooling effect, in addition to the anti-inflammatory and vein-sealing effect of the red vine leaf extract, briefly helps to relieve the pain of CVI.

By “solvents” are meant within the scope of the present invention any cosmetically and pharmaceutically acceptable agents that are suitable for dissolving ingredients of the sprayable composition. Preferred solvents are water, aliphatic alcohols such as e.g. ethanol, isopropanol, glycols such as e.g. glycerol, propyleneglycol or fatty acid esters, such as e.g. isopropylmyristate, oleyl oleate and high molecular solvents, such as e.g. polyethyleneglycols, and the mixtures thereof. A preferred solvent is water.

The term “skin-care substances” within the scope of the present invention refers to all cosmetically and pharmaceutically acceptable agents which when applied directly have beneficial effects on the appearance and health of the human skin. Skin moisture regulators such as e.g. propyleneglycol, urea or panthenol, substances that improve the absorption of substances through the skin (penetration accelerators), such as e.g. urea and propyleneglycol, and substances that improve skin regeneration, such as e.g. panthenol, and mixtures thereof, are preferred. Particularly preferred skin-care substances are urea and panthenol. Preferred penetration accelerators are urea and propyleneglycol.

By “adjuvants” are meant, within the scope of the present invention, all cosmetically and pharmaceutically acceptable agents that improve the physical properties of the sprayable composition or its stability. These are preferably stabilisers, such as e.g. aminomethyl-propanol, preservatives such as e.g. methylparaben, emulsifiers, such as e.g. PEG-40 hydrogenated castor oil, viscosity adjusters such as e.g. isopropylmyristate and gel-forming agents, such as e.g. crosslinked and uncrosslinked polyacrylates, carboxymethylcellulose hydroxyethylcellulose, methylcellulose. Other adjuvants are colours and perfumes. Adjuvants such as isopropylmyristate, among other things, help the composition to spread better over the skin, i.e. to be distributed better.

Particularly preferred embodiments of the composition according to the present invention are so-called spray gels. These have a viscosity in the range from 15000 to 70000 mPas, preferably at least 20000 mPas, particularly preferably at least 50000 mPas and particularly preferably at least 60000 mPas, in each case at 20° C., and remain on the skin longer than lower-viscosity compositions and are therefore able to develop their effect on the affected areas of skin for longer periods.

The sprayable composition is used according to the invention in a spray container, from which it is sprayed by a spray device, typically a spray nozzle, onto the user's skin Suitable spray containers with suitable spray devices are available on the market. Particularly suitable spray containers for spray gels are the spray containers of commercially obtainable products such as Hansaplast spray plaster, Polysport spray plaster, Scholl gel plaster, Flint spray dressing, Hansaplast Aktiv Gel plaster, Compeed Liquid spray plaster or Softivel spray plaster. According to the present invention the composition is held in the spray container under a pressure in the range from 1.5 to 20 bar, preferably 2 to 15 bar, preferably 3 to 12 bar and particularly preferably 4 to 8 bar, in each case at 20° C.

A particularly preferred embodiment of the present invention is a hydrophilic spray gel containing red vine leaf extract with propellants that are soluble in the aqueous medium under pressure, such as for example dimethylether, and preferably also containing urea, propyleneglycol, isopropylmyristate and panthenol as additional main ingredients.

The following is a particularly preferred embodiment of the sprayable composition according to the present invention:

| | | |
|---|---|---|
| purified water | 66.49 | wt. % |
| dimethyl ether | 26.00 | wt. % |
| urea | 2.91 | wt. % |
| 1,2 propylene glycol | 1.74 | wt. % |
| liquid extract of red | 1.00 | wt. % |
| vine leaf, extracting | | |
| agents: 60% ethanol in | | |
| water; 1:1 mixture | | |
| with extracting agent | | |
| isopropyl myristate | 0.58 | wt. % |
| D-panthenol 75 W | 0.44 | wt. % |
| Carbomer 940 | 0.21 | wt. % |
| (Synthalen K) | | |
| aminomethyl propanol | 0.20 | wt. % |
| (AMP Ultra PC 1000) | | |
| PEG-40 Hydrogenated | 0.17 | wt. % |
| Castor Oil (Cremophor | | |
| RH 40) | | |
| methylparaben | 0.07 | wt. % |
| perfume | 0.17 | wt. % |

1. A sprayable composition containing red vine leaf extract, the composition further comprising water and a propellant that is miscible with water under elevated pressure.

2. The composition according to claim 1, wherein the extract for obtaining the red vine leaf extract is selected from among the group consisting of water, ethanol and mixtures thereof.

3. The composition according to claim 2, wherein the red vine leaf extract is obtained by extracting leaves of the species vitis vinifera L.

4. The composition according to claim 1, wherein the red vine leaf extract has been prepared by a process comprising the following steps:
(a) collecting red vine leaves at a time when the flavonoid content has reached an optimum level;
(b) drying and comminuting the leaves;
(c) cutting the leaves into pieces;
(d) extracting the shredded leaves with water or ethanol or mixtures thereof at elevated temperature for 6 to 10 hours;
(e) optionally concentrating the extract obtained.

5. The composition according to claim 3, wherein the composition contains red vine leaf extract in a concentration of 0.1 wt.-% to 15 wt.-%, based on the dry extract and the total mass of the composition.

6. The composition according to claim 5, wherein the red vine leaf extract contains flavonoids in the range from 1 to 20 wt.-%, based on the dry mass of the red vine leaf extract.

7. The composition according to claim 6, wherein the composition contains one or more other ingredients, selected from among propellants, solvents, skin-care substances and adjuvants.

8. The composition according to claim 7, wherein the propellant comprises at least one highly volatile substance with a vapour pressure in the range from 1 to 10 bar, at 20° C.

9. The composition according to claim 8, containing at least 1 wt.-%, based on the total mass of the composition.

10. The composition according to claim 8, wherein the propellant is selected from among propellant gases that can be liquefied under elevated pressure and are miscible with water under elevated pressure.

11. The composition according to claim 10, wherein the propellant is dimethylether.

12. The composition according to claim 11, wherein the solvents are selected from among water, ethanol, isopropanol, glycerol, propyleneglycol, fatty acid esters, oleyl oleates and polyethyleneglycols and mixtures thereof.

13. The composition according to claim 12, wherein the skin-care substances are selected from among from among propyleneglycol, urea, panthenol and the mixtures thereof.

14. The composition according to claim 12, wherein the adjuvants are selected from among aminomethyl-propanol, methylparaben, PEG-40 hydrogenated castor oil, isopropylmyristate, crosslinked and uncrosslinked polyacrylates.

15. The composition according to claim 14, wherein the composition is a spray gel with a viscosity in the range from 15000 to 70000 mPas, at 20° C. and 1 bar.

16-17. (canceled)

18. The composition according to claim 3 wherein
the composition contains red vine leaf extract in a concentration of 0.3 wt.-% to 10 wt.-% based on the dry extract and the total mass of the composition;
the red vine leaf extract contains flavonoids in the range from 2 to 10 wt.-% based on the dry mass of the red vine leaf extract; and
wherein the propellant comprises at least one highly volatile substance with a vapour pressure in the range from 3 to 8 bar at 20° C.
